(19) United States
(12) Patent Application Publication
(10) Pub. No.: US 2022/0160597 A1
(43) Pub. Date: May 26, 2022

(54) DENTAL CALCULUS REMOVAL

(71) Applicant: Howard Frysh, Dallas, TX (US)

(72) Inventor: Howard Frysh, Dallas, TX (US)

(21) Appl. No.: 17/455,780

(22) Filed: Nov. 19, 2021

Related U.S. Application Data
(60) Provisional application No. 63/116,590, filed on Nov. 20, 2020.

(51) Int. Cl. A61K 8/34; A61K 8/60; A61Q 11/00; A61C 3/00; A61C 17/20

(52) U.S. Cl. CPC A61K 8/345; A61K 8/60; A61Q 11/00; A61K 2800/30; A61C 17/20; A61K 2800/33; A61C 3/00

(57) ABSTRACT

Provided herein are aqueous compositions useful for softening and loosening dental calculus deposits. The compositions include high concentrations of sorbitol and minimal or no other components. Also provided are methods for using the provided compositions in the removal of calculus from a tooth surface.

### CROSS-REFERENCE TO RELATED APPLICATIONS

The present application claims priority to U.S. Provisional Application No. 63/116,590 filed Nov. 20, 2020, the full disclosure of which is incorporated by reference in its entirety for all purposes.

### BACKGROUND

Calculus, also known as tartar, is a form of hardened dental plaque caused by the precipitation of minerals from saliva and gingival crevicular fluid. The rough and hardened surface formed by deposits of calculus provide an environment well-suited for supporting the growth of various oral bacterial species. Colonies of these bacteria metabolize sugars and starches to release acids causing complications that include cavities and the erosion of tooth enamel. The promotion of bacterial growth through calculus buildup is also a significant cause of periodontal disease, which has been associated with health risks that extend beyond gingivitis, and gum loss, and may even be a factor in an increased risk of heart disease.

Calculus removal is typically beyond the scope of home personal dental care, requiring instead the attention of dental hygienists and dentists during dental care office visits. These dental professionals apply tools and techniques such as scaling in a debridement process for removing calculus from the visible surfaces of affected teeth, as well as tooth surfaces below the gumline. Despite advances in scaling and root planning methodologies, however, a significant amount of calculus can remain unremoved after these interventions.

Moreover, ultrasonic scalers can carry additional risks related to their use of a water spray to cool their rapidly vibrating scaler tips, flush debris from the treated tooth, create bubbles that can disrupt bacteria, and irrigate gums during debridement. While these functions can provide important improvements over hand scaling not involving water sprays, increasing the effectiveness in removing calculus and infectious materials from the tooth surface of a patient, they also can create aerosols of the removed materials. These aerosols can permeate into and linger within the treatment room or suite, and could serve as a delivery mechanism for transferring infections from one patient to others or to dental care professionals. Such concerns are particularly relevant during, for example, the COVID-19 pandemic, and can lead many calculus treatment procedures to opt for less effective hand scaling procedures to prevent aerosol formations.

In view of these observations and results, there is a need in the art for improved materials and methods that loosen, soften, or dissolve calculus deposits, thereby rendering the deposits more easily removed from tooth surfaces. The present disclosure addresses this need and provides associated and other advantages.

### BRIEF SUMMARY

The present disclosure generally relates to compositions that, when employed for example in the loosening, softening, or dissolving of calculus present on a tooth surface, provide several advantageous improvements. For example, it is beneficial in the removal of dental plaque such as calculus for the rigid structure of the plaque to be broken down, and for its attachment to a tooth surface to be weakened. Conventional approaches to calculus removal rely exclusively or primarily on the application of mechanical forces by dental hygienists or dentists in scraping or fracturing calculus deposits to pry the deposits from the surfaces to which they are affixed. These approaches can permit a substantial fraction of the targeted calculus to remain on a patient's tooth, can cause patient discomfort associated with the sharpness of the instruments used or the strength of the forces applied, and can result in practitioner stress injuries associated with the pressure and repetition of scaling.

To address the above challenges, the inventor has now developed a different approach to calculus removal in which a composition, e.g., a liquid solution having a high sorbitol concentration approaching saturation, is brought into contact with calculus deposits. The particular selection of components and concentrations in the composition surprisingly causes calculus to begin to lose its rigidity and solidity, and to start to release from tooth surfaces. As a result, removal of calculus, through for example, subsequent scaling or other debridement processes, can be both easier and more effective.

In one aspect, the disclosure is to a composition for the removal of dental calculus, wherein the composition consists essentially of sorbitol, glycerol, and water. The concentration of sorbitol in the composition ranges from 50 wt % to 85 wt %. The concentration of glycerol in the composition is less than 10 wt %.

In another aspect, the disclosure is to a composition for the removal of dental calculus, wherein the composition consists of sorbitol, glycerol, and water. The concentration of sorbitol in the composition ranges from 50 wt % to 85 wt %. The concentration of glycerol in the composition is less than 10 wt %.

In another aspect, the disclosure is to a method for removing dental calculus from a tooth surface. The method includes contacting the tooth surface with any of the compositions disclosed herein. The method further includes scaling dental calculus from the tooth surface.

### DETAILED DESCRIPTION

Sorbitol is an ingredient in many dental care formulations, used primarily due to its resistance to metabolism by most bacteria, and in particular those species most prevalent in oral environments. Because sorbitol cannot provide a nutritional source for these bacteria, its presence in toothpastes and mouthwashes, as well as in food and beverages, generally does not promote tooth decay. Sorbitol has also found use in these products for its various other beneficial properties, including as a humectant, a flavorant, and a food softener.

The inventor has now surprisingly demonstrated that high concentrations of dissolved sorbitol, e.g., concentrations approaching or at saturation or supersaturation levels, particularly when present with few or no additional solution components, are effective in improving calculus removal. Without being bound by a particular theory, it is believed that the surprising ability of concentrated sorbitol to soften calculus and loosen it from tooth surfaces can at least partially be attributed to the chelating and hygroscopic characteristics possessed by sorbitol. For example, sorbitol may act to instigate the breakdown of calculus by forming chelation complexes with mineral components of calculus deposits. Sorbitol complexes have been characterized with metals including copper, molybdenum, and boron (T. Allscher, P. Klufers & P. Mayer, Glycoscience (2008)). The chelating ability of sorbitol has been recognized and applied elsewhere to, for example, the complexing of calcium delivered in plant fertilization sprays (P. Li et al., 16 Am. J. Biochem. Biotechnol. 86 (2020)). In addition, the high hygroscopicity of sorbitol has been shown to promote degradative reactions such as hydrolyses (W. L. Chen, D. W. Guo, Y. Y. Shen, S. R. Guo & K. P. Ruan, 74 Indian J. Pharm. Sci. 527 (2012)). As a result, sorbitol may act to attract and maintain a chemical environment proximate to the calculus that supports beneficial and advantageous hydrolytic calculus degradation reactions.

Additionally, the inventor has recognized that the desired calculus softening and loosening abilities of sorbitol are significantly improved when additional solution components are not present. Without being bound to a particular theory, it is believed that the inclusion of other components in the concentrated sorbitol solution can lead to interference with, for example, the chelating and hygroscopic properties discussed above. As an example, the presence in the sorbitol solution of a metal such as fluoride, or of a metal-containing compound, can introduce an alternative chelation complex partner in competition with the minerals present in the targeted calculus. This competition can reduce the fraction of sorbitol acting on the calculus, leading to a decrease in calculus softening efficacy. Importantly, this observation has not been appreciated or taken advantage of in previous dental care formulations which either include only a low level of sorbitol, or add multiple other components as active or inactive ingredients.

### Compositions

In one aspect of the present disclosure, a composition is disclosed. The composition includes a high concentration of a sugar alcohol. In some embodiments, the composition is an aqueous solution of a sugar alcohol. Compositions including a sugar alcohol at concentrations approaching their solubility have been demonstrated by the inventor to be effective in softening and loosening dental calculus. In some embodiments, the composition comprises a sugar alcohol and water. In some embodiments, the composition consists essentially of a sugar alcohol and water. In some embodiments, the composition consists of a sugar alcohol and water. Preferably, the sugar alcohol of the composition is sorbitol.

The concentration of sorbitol in the provided composition can be selected to be high enough to effectively soften and loosen dental calculus for improving its removal, and low enough to not exceed the sorbitol solubility under conditions typical of manufacturing, shipping, storage, and/or application environments. The concentration of sorbitol can be selected to approach the solubility limit of sorbitol in water, or to create a supersaturated sorbitol solution. The sorbitol concentration in the composition can be, for example, between 50 wt % and 85 wt %, e.g., between 50 wt % and 71 wt %, between 53.5 wt % and 74.5 wt %, between 57 wt % and 78 wt %, between 60.5 wt % and 81.5 wt %, or between 64 wt % and 85 wt %. In terms of upper limits, the sorbitol concentration in the composition can be less than 85 wt %, e.g., less than 81.5 wt %, less than 78 wt %, less than 74.5 wt %, less than 71 wt %, less than 67.5 wt %, less than 64 wt %, less than 60.5 wt %, less than 57 wt %, or less than 53.5 wt %. In terms of lower limits, the sorbitol concentration in the composition can be greater than 50 wt %, e.g., greater than 53.5 wt %, greater than 57 wt %, greater than 60.5 wt %, greater than 64 wt %, greater than 67.5 wt %, greater than 71 wt %, greater than 74.5 wt %, greater than 78 wt %, or greater than 81.5 wt %. Higher sorbitol concentrations, e.g., greater than 85 wt %, and lower sorbitol concentrations, e.g., less than 50 wt %, are also contemplated.

In some embodiments, the composition further includes glycerol. Compositions including glycerol have been demonstrated by the inventor to have improved flow and stability in certain applications. In some embodiments, the composition comprises sorbitol, glycerol, and water. In some embodiments, the composition consists essentially of sorbitol, glycerol, and water. In some embodiments, the composition consists of sorbitol, glycerol, and water.

The concentration of glycerol in the provided composition can be selected to be high enough to improve flow and stability properties of the composition, and low enough to maintain the liquid characteristic of the composition and not interfere with the efficacy of the composition sorbitol. The glycerol concentration in the composition can be, for example, between 0 and 10 wt %, e.g., between 0 and 1.6 wt %, between 0.16 wt % and 2.5 wt %, between 0.25 wt % and 4 wt %, between 0.4 wt % and 6.3 wt %, or between 0.63 wt % and 10 wt %. In terms of upper limits, the glycerol concentration in the composition can be less than 10 wt %, e.g., less than 6.3 wt %, less than 4 wt %, less than 2.5 wt %, less than 1.6 wt %, less than 1 wt %, less than 0.63 wt %, less than 0.4 wt %, less than 0.25 wt %, or less than 0.16 wt %. In terms of lower limits, the glycerol concentration in the composition can be greater than 0.16 wt %, e.g., greater than 0.25 wt %, greater than 0.4 wt %, greater than 0.63 wt %, greater than 1 wt %, greater than 1.6 wt %, greater than 2.5 wt %, greater than 4 wt %, or greater than 6.3 wt %. Higher glycerol concentrations, e.g., greater than 10 wt %, are also contemplated.

In some embodiments, the mass ratio of the sugar alcohol, e.g., sorbitol, in the composition to the glycerol in the composition ranges from 5:1 to 50:1, e.g., from 5:1 to 20:1, from 6.3:1 to 25:1, from 7.9:1 to 32:1, from 10:1 to 40:1, or from 13:1 to 50:1. In terms of upper limits, the mass ratio of the sorbitol to the glycerol in the composition can be less than 50:1, e.g., less than 40:1, less than 32:1, less than 25:1, less than 20:1, less than 16:1, less than 13:1, less than 10:1, less than 7.9:1, or less than 6.3:1. In terms of lower limits, the mass ratio of the sorbitol to the glycerol in the composition can be greater than 5:1, e.g., greater than 6.3:1, greater than 7.9:1, greater than 10:1, greater than 13:1, greater than 16:1, greater than 20:1, greater than 25:1, greater than 32:1, or greater than 40:1. Higher mass ratios, e.g., greater than 50:1, and lower mass ratios, e.g., less than 5:1, are also contemplated.

In some embodiments, the composition includes only one sugar alcohol, e.g., sorbitol. As used herein, a statement that the composition does not comprise an additional sugar alcohol is intended to include embodiments having an unintentional presence of a trace amount of a second sugar alcohol, e.g., at a concentration on the order of 1 ppm or 1 ppb, and is intended to exclude embodiments having an intentional amount and/or a higher amount of the second sugar alcohol. The lack of additional sugar alcohols in the composition is preferred in certain applications to prevent interference with the efficacy of the composition sugar alcohol in softening and loosening dental calculus. The lack of additional sugar alcohols can also in certain applications reduce composition formulation costs, increase composition stability, and provide other benefits. In some embodiments, the composition does not include xylitol. In some embodiments, the composition does not include mannitol. In some embodiments, the composition does not include maltitol. In some embodiments, the composition does not include a sugar alcohol derivative, such as, for example, propylene glycol or polyethylene glycol.

In some embodiments, the composition further includes mannose, e.g., D-mannose or L-mannose. Compositions including mannose have been demonstrated by the inventor to reduce crystallization of sorbitol, In some embodiments, the composition comprises sorbitol, glycerol, mannose, and water. In some embodiments, the composition consists essentially of sorbitol, glycerol, mannose, and water. In some embodiments, the composition consists of sorbitol, glycerol, mannose, and water.

The concentration of mannose in the provided composition can be, for example, between 0 and 10 wt %, e.g., between 0 and 1.6 wt %, between 0.16 wt % and 2.5 wt %, between 0.25 wt % and 4 wt %, between 0.4 wt % and 6.3 wt %, or between 0.63 wt % and 10 wt %. In terms of upper limits, the mannose concentration in the composition can be less than 10 wt %, e.g., less than 6.3 wt %, less than 4 wt %, less than 2.5 wt %, less than 1.6 wt %, less than 1 wt %, less than 0.63 wt %, less than 0.4 wt %, less than 0.25 wt %, or less than 0.16 wt %. In terms of lower limits, the mannose concentration in the composition can be greater than 0.16 wt %, e.g., greater than 0.25 wt %, greater than 0.4 wt %, greater than 0.63 wt %, greater than 1 wt %, greater than 1.6 wt %, greater than 2.5 wt %, greater than 4 wt %, or greater than 6.3 wt %. Higher mannose concentrations, e.g., greater than 10 wt %, are also contemplated.

In some embodiments, the composition does not include a metal. As used herein, a statement that the composition does not comprise a metal is intended to include embodiments having an unintentional presence of a trace amount of a metal, e.g., at a concentration on the order of 1 ppm or 1 ppb, and is intended to exclude embodiments having an intentional amount and/or a higher amount of the metal. The lack of a metal in the composition is preferred in certain applications to prevent interference with the efficacy of the composition sugar alcohol in softening and loosening dental calculus. The lack of a metal can also in certain applications reduce composition formulation costs, increase composition stability, and provide other benefits. In some embodiments, the composition does not include fluoride or a fluoride-containing compound. In some embodiments, the composition does not include calcium or a calcium-containing compound. In some embodiments, the composition does not include magnesium or a magnesium-containing compound. In some embodiments, the composition does not include zinc or a zinc-containing compound. In some embodiments, the composition does not include potassium or a potassium-containing compound. In some embodiments, the composition does not include aluminum or an aluminum-containing compound.

In some embodiments, the composition does not include a chelating agent other than the sugar alcohol, e.g., sorbitol. As used herein, a statement that the composition does not comprise a second chelating agent is intended to include embodiments having an unintentional presence of a trace amount of a second chelating agent, e.g., at a concentration on the order of 1 ppm or 1 ppb, and is intended to exclude embodiments having an intentional amount and/or a higher amount of the second chelating agent. The lack of additional chelating agents in the composition is preferred in certain applications to prevent interference with the efficacy of the composition sugar alcohol in softening and loosening dental calculus. The lack of additional chelating agents can also in certain applications reduce composition formulation costs, increase composition stability, and provide other benefits. In some embodiments, the composition does not include ethylenediaminetetraacetic acid (EDTA), or a salt or complex thereof. In some embodiments, the composition does not include sodium polyphosphate or sodium tripolyphosphate (STPP). In some embodiments, the composition does not include tetrasodium pyrophosphate (TSPP).

In some embodiments, the composition does not include a humectant other than the sugar alcohol, e.g., sorbitol. As used herein, a statement that the composition does not comprise a second humectant is intended to include embodiments having an unintentional presence of a trace amount of a second humectant, e.g., at a concentration on the order of 1 ppm or 1 ppb, and is intended to exclude embodiments having an intentional amount and/or a higher amount of the second humectant. The lack of additional humectants in the composition is preferred in certain applications to prevent interference with the efficacy of the composition sugar alcohol in softening and loosening dental calculus. The lack of additional humectants can also in certain applications reduce composition formulation costs, increase composition stability, and provide other benefits. In some embodiments, the composition does not include propylene glycol.

In some embodiments, the composition does not include a surfactant. As used herein, a statement that the composition does not comprise a surfactant is intended to include embodiments having an unintentional presence of a trace amount of a surfactant, e.g., at a concentration on the order of 1 ppm or 1 ppb, and is intended to exclude embodiments having an intentional amount and/or a higher amount of the surfactant. The lack of a surfactant in the composition is preferred in certain applications to prevent interference with the efficacy of the composition sugar alcohol in softening and loosening dental calculus. The lack of a surfactant can also in certain applications reduce composition formulation costs, increase composition stability, and provide other benefits. In some embodiments, the composition does not include sodium lauryl sulfate (SLS). In some embodiments, the composition does not include a poloxomer such as, for example, a commercially available SYNPERONIC™, PLURONIC®, or KOLLIPHOR® compound.

In some embodiments, the composition does not include an abrasive. As used herein, a statement that the composition does not comprise an abrasive is intended to include embodiments having an unintentional presence of a trace amount of an abrasive, e.g., at a concentration on the order of 1 ppm or 1 ppb, and is intended to exclude embodiments having an intentional amount and/or a higher amount of the abrasive. The lack of an abrasive in the composition is preferred in certain applications to prevent interference with the efficacy of the composition sugar alcohol in softening and loosening dental calculus. The lack of an abrasive can also in certain applications reduce composition formulation costs, increase composition stability, and provide other benefits. In some embodiments, the composition does not include silica, or a compound or complex thereof. In some embodiments, the composition does not include a phosphate salt. In some embodiments, the composition does not include a zeolite or a complex thereof. In some embodiment, the composition does not include a carbonate or a complex thereof. In some embodiments, the composition does not include an oxide or a complex thereof. In some embodiments, the composition does not include aluminum hydroxide. In some embodiments, the composition does not include hydroxyapatite.

In some embodiments, the composition does not include an antibacterial agent other than the sugar alcohol, e.g., sorbitol. As used herein, a statement that the composition does not comprise a second antibacterial agent is intended to include embodiments having an unintentional presence of a trace amount of a second antibacterial agent, e.g., at a concentration on the order of 1 ppm or 1 ppb, and is intended to exclude embodiments having an intentional amount and/or a higher amount of the second antibacterial agent. The lack of additional antibacterial agents in the composition is preferred in certain applications to prevent interference with the efficacy of the composition sugar alcohol in softening and loosening dental calculus. The lack of additional antibacterial agents can also in certain applications reduce composition formulation costs, increase composition stability, and provide other benefits. In some embodiments, the composition does not include triclosan. In some embodiments, the composition does not include chlorhexidine. In some embodiments, the composition does not include an essential oil such as, for example, menthol, thymol, methyl salicylate, or eucalyptol.

In some embodiments, the composition does not include zinc chloride. In some embodiments, the composition does not include lactoperoxidase. In some embodiments, the composition does not include lysozyme. In some embodiments, the composition does not include lactoferrin. In some embodiments, the composition does not include methyl salicylate.

In some embodiments, the composition does not include an acid or a base. As used herein the terms “acid” and “base” refer to compounds having a pKa less than 10, and compounds having a pKb less than 10, respectively. As used herein, a statement that the composition does not comprise an acid or base is intended to include embodiments having an unintentional presence of a trace amount of an acid or base, e.g., at a concentration on the order of 1 ppm or 1 ppb, and is intended to exclude embodiments having an intentional amount and/or a higher amount of the acid or base. The lack of an acid or base in the composition is preferred in certain applications to prevent interference with the efficacy of the composition sugar alcohol in softening and loosening dental calculus. The lack of an acid or base can also in certain applications reduce composition formulation costs, increase composition stability, and provide other benefits. In some embodiments, the composition does not include sodium hydroxide. In some embodiments, the composition does not include sodium bicarbonate. In some embodiments, the composition does not include phosphoric acid. In some embodiments, the composition does not include citric acid. In some embodiments, the composition does not include benzoic acid. In some embodiments, the composition includes a minimal nonzero amount, e.g., less than 1 wt %, of a base, e.g., sodium hydroxide. In embodiments, in which the composition has the form of a gel, the minimal amount of base can assist in the formation of the gel.

In some embodiments, the composition does not include a whitening agent. As used herein, a statement that the composition does not comprise a whitening agent is intended to include embodiments having an unintentional presence of a trace amount of a whitening agent, e.g., at a concentration on the order of 1 ppm or 1 ppb, and is intended to exclude embodiments having an intentional amount and/or a higher amount of the whitening agent. The lack of a whitening agent in the composition is preferred in certain applications to prevent interference with the efficacy of the composition sugar alcohol in softening and loosening dental calculus. The lack of a whitening agent can also in certain applications reduce composition formulation costs, increase composition stability, and provide other benefits. In some embodiments, the composition does not include a peroxide compound.

In some embodiments, the composition does not include a flavorant other than the sugar alcohol, e.g., sorbitol. As used herein, a statement that the composition does not comprise a second flavorant is intended to include embodiments having an unintentional presence of a trace amount of a second flavorant, e.g., at a concentration on the order of 1 ppm or 1 ppb, and is intended to exclude embodiments having an intentional amount and/or a higher amount of the second flavorant. The lack of additional flavorants in the composition is preferred in certain applications to prevent interference with the efficacy of the composition sugar alcohol in softening and loosening dental calculus. The lack of additional flavorants can also in certain applications reduce composition formulation costs, increase composition stability, and provide other benefits. In some embodiments, the composition does not include an essential oil. In some embodiments, the composition does not include a sweetener such as, for example, a saccharin compound

In some embodiments, the composition does not includes a colorant. As used herein, a statement that the composition does not comprise a colorant is intended to include embodiments having an unintentional presence of a trace amount of a colorant, e.g., at a concentration on the order of 1 ppm or 1 ppb, and is intended to exclude embodiments having an intentional amount and/or a higher amount of the colorant. The lack of a colorant in the composition is preferred in certain applications to prevent interference with the efficacy of the composition sugar alcohol in softening and loosening dental calculus. The lack of a colorant can also in certain applications reduce composition formulation costs, increase composition stability, and provide other benefits. In some embodiments, the composition does not include an artificial coloring dye or compound. In some embodiments, the composition does not include a natural coloring dye or compound.

In some embodiments, the composition does not include a solvent or carrier other than water. As used herein, a statement that the composition does not comprise an additional solvent or carrier is intended to include embodiments having an unintentional presence of a trace amount of an additional solvent or carrier, e.g., at a concentration on the order of 1 ppm or 1 ppb, and is intended to exclude embodiments having an intentional amount and/or a higher amount of the additional solvent or carrier. The lack of an additional solvent or carrier in the composition is preferred in certain applications to prevent interference with the efficacy of the composition sugar alcohol in softening and loosening dental calculus. The lack of an additional solvent or carrier can also in certain applications reduce composition formulation costs, increase composition stability, and provide other benefits. In some embodiments, the composition does not include ethanol.

In some embodiments, the composition includes a small nonzero amount, e.g., less than 1 wt % each, of one or more preservatives. Preservatives suitable for use with the provided composition include, for example, benzoic acid, benzyl alcohol, ethyl paraben, methyl paraben, propyl paraben, sodium benzoate, sorbic acid, salts thereof, and combinations thereof. In some embodiments, the composition does not include a preservative. As used herein, a statement that the composition does not comprise a preservative is intended to include embodiments having an unintentional presence of a trace amount of a preservative, e.g., at a concentration on the order of 1 ppm or 1 ppb, and is intended to exclude embodiments having an intentional amount and/or a higher amount of the preservative.

In some embodiments, the composition is essentially free of one component selected from the list including sugar alcohols other than sorbitol, chelating agents other than sorbitol, humectants other than sorbitol, antibacterial agents other than sorbitol, flavoring agents other than sorbitol, metals, surfactants, abrasives, acids, bases, whitening agents, and colorants. In some embodiments, the composition is essentially free of two components selected from the list including sugar alcohols other than sorbitol, chelating agents other than sorbitol, humectants other than sorbitol, antibacterial agents other than sorbitol, flavoring agents other than sorbitol, metals, surfactants, abrasives, acids, bases, whitening agents, and colorants. In some embodiments, the composition is essentially free of three components selected from the list including sugar alcohols other than sorbitol, chelating agents other than sorbitol, humectants other than sorbitol, antibacterial agents other than sorbitol, flavoring agents other than sorbitol, metals, surfactants, abrasives, acids, bases, whitening agents, and colorants. In some embodiments, the composition is essentially free of four or more, e.g., five or more, six or more, seven or more, eight or more, nine or more, or ten or more, components selected from the list including sugar alcohols other than sorbitol, chelating agents other than sorbitol, humectants other than sorbitol, antibacterial agents other than sorbitol, flavoring agents other than sorbitol, metals, surfactants, abrasives, acids, bases, whitening agents, and colorants. In some embodiments, the composition is essentially free of each component selected from the list including sugar alcohols other than sorbitol, chelating agents other than sorbitol, humectants other than sorbitol, antibacterial agents other than sorbitol, flavoring agents other than sorbitol, metals, surfactants, abrasives, acids, bases, whitening agents, and colorants.

In some embodiments, the provided composition is a gel. As used herein, the term “gel” refers to a soft, solid or solid-like material having a liquid component and exhibiting no flow when in a steady state. Gel compositions can provide an advantage in some applications by increasing the ease with which the composition can be handled, stored, and/or delivered to a tooth surface. Gelling agents suitable for use with the provided composition include, for example, xanthan gum.

In some embodiments, the provided composition is a liquid. As used herein the term “liquid” refers to a substantially incompressible fluid material that is substantially or entirely free from insoluble material. Liquid compositions can provide an advantage in some applications by decreasing the volume of the composition and the improving the suitability of the composition for being sprayed or painted onto the surface of a tooth. Unlike other dental care formulations that necessarily include additional components, e.g., insoluble or cross-linking components, and that are intended for dental care applications exclusively as pastes or gels, the provided compositions advantageously can have a liquid form.

### Methods

Another aspect of the present disclosure is a method of removing dental calculus from at least a portion of a surface of a tooth. The method includes contacting the tooth surface, e.g., the portion of the tooth surface, with any of the compositions provided herein. In some embodiments, the method includes contacting the tooth surface with a composition, e.g., a liquid composition, consisting essentially of sorbitol at a concentration in the composition ranging from 50 wt % to 85 wt %, glycerol at a concentration in the composition less than 10 wt %, and water. In some embodiments, the method includes contacting the tooth surface with a composition, e.g., a liquid composition, consisting of sorbitol at a concentration in the composition ranging from 50 wt % to 85 wt %, glycerol at a concentration in the composition less than 10 wt %, and water.

The method further includes, subsequent to the contacting of the tooth surface with the provided composition, scaling dental calculus from the tooth surface that was contacted with the composition. Because of the advantages discussed above, dental calculus on the contacted tooth surface will be more easily removed than dental calculus could otherwise be removed from a tooth surface not contacted by the provided composition. This is because of the ability of the provided composition to soften and loosen the calculus.

In some embodiments, the scaling is performed using a hand scaler. As used herein, the term “hand scaler” refers to a handheld, typically non-electric, dental instrument having a scraping edge and used in the prophylactic and periodontal care of teeth. The term also includes periodontal curettes, jaquettes, hoes, files, and chisels.

In some embodiments, the scaling is performed using an ultrasonic scaler. As used herein, the term “ultrasonic scaler” refers to an electric dental instrument having an oscillating tip that vibrates at high speeds, and typically further having a water spray directed towards the end of the oscillating tip. Ultrasonic scalers in certain applications can be faster and less irritating to a patient than hand scalers, but also can create aerosols capable of spreading pathogens when a patient is carrying an infectious disease. In some embodiments, the use of the provided method allows procedures typically performed using ultrasonic scalers to be replaced with alternative procedures performed using hand scalers, thereby reducing the risk of aerosol formation without sacrificing patient comfort or calculus removal efficiency and effectiveness.

In some embodiments, the method further includes, prior to the contacting of the tooth surface with the provided composition, heating the composition, e.g., to a temperature higher than ambient room temperature. While the use of the composition at room temperatures or lower can be effective in calculus removal, in some embodiments, the contacting of a tooth surface with such a heated composition increases the degree to which the composition is able to soften and loosen dental calculus on the tooth surface. This increased softening and loosening can beneficially result in more efficient removal of calculus from the tooth surface during the subsequent scaling step of the provided method.

The temperature of the provided composition during the contacting of the tooth surface with the composition can be selected to be high enough to increase the calculus softening and loosening efficacy of the composition, and low enough to not significantly increase composition volatility or patient discomfort. The composition temperature can range, for example, from 30° C. to 50° C., e.g., from 30° C. to 42° C., from 32° C. to 44° C., from 34° C. to 46° C., from 36° C. to 48° C., or from 38° C. to 50° C. In terms of upper limits, the composition temperature can be less than 50° C., e.g., less than 48° C., less than 46° C., less than 44° C., less than 42° C., less than 40° C., less than 38° C., less than 36° C., less than 34° C., or less than 32° C. In terms of lower limits, the composition temperature can be greater than 30° C., e.g., greater than 32° C., greater than 34° C., greater than 36° C., greater than 38° C., greater than 40° C., greater than 42° C., greater than 44° C., greater than 46° C., or greater than 48° C. Higher composition temperatures, e.g., greater than 50° C., and lower composition temperatures, e.g., less than 30° C., are also contemplated.

In some embodiments, the scaling of dental calculus from the tooth surface begins immediately after contacting the tooth surface with the provided composition. In some embodiments, it is preferred to delay the scaling to allow the composition to soften the calculus on the tooth surface prior to the calculus removal. The delay between the contacting and the scaling can be, for example, from 1 minute to 30 minutes, e.g., from 1 minute to 8 minutes, from 1.5 minutes to 11 minutes, from 2 minutes to 15 minutes, from 3 minutes, to 21 minutes, or from 4 minutes to 30 minutes. In terms of upper limits, the scaling can be begun less than 30 minutes, e.g., less than 21 minutes, less than 15 minutes, less than 11 minutes, less than 8 minutes, less than 5 minutes, less than 4 minutes, less than 3 minutes, less than 2 minutes, or less than 1.5 minutes after the contacting. In terms of lower limits, the scaling can be begun more than 1 minute, e.g., more than 1.5 minutes, more than 2 minutes, more than 3 minutes, more than 4 minutes, more than 5 minutes, more than 8 minutes, more than 11 minutes, more than 15 minutes, or more than 21 minutes after the contacting. Longer delays, e.g., more than 30 minutes, and shorter delays, e.g., less than 1 minutes, are also contemplated.

### EMBODIMENTS

The following embodiments are contemplated. All combinations of features and embodiment are contemplated.

Embodiment 1: A composition for the removal of dental calculus, the composition consisting essentially of: sorbitol, wherein the concentration of sorbitol in the composition ranges from 50 wt % to 85 wt %; glycerol, wherein the concentration of glycerol in the composition is less than 10 wt %; and water; wherein the composition is a liquid.

Embodiment 2: An embodiment of embodiment 1, wherein the mass ratio of the sorbitol to the glycerol ranges from 5:1 to 50:1.

Embodiment 3: An embodiment of embodiment 1 or 2, wherein the composition further comprises mannose, and wherein the concentration of mannose in the composition is less than 10 wt %.

Embodiment 4: An embodiment of any of the embodiments of embodiment 1-3, wherein the composition does not comprise an additional sugar alcohol.

Embodiment 5: An embodiment of any of the embodiments of embodiment 1-4, wherein the composition does not comprise a metal.

Embodiment 6: An embodiment of any of the embodiments of embodiment 1-5, wherein the composition does not comprise an additional chelating agent.

Embodiment 7: An embodiment of any of the embodiments of embodiment 1-6, wherein the composition does not comprise an additional humectant.

Embodiment 8: An embodiment of any of the embodiments of embodiment 1-7, wherein the composition does not comprise a surfactant.

Embodiment 9: An embodiment of any of the embodiments of embodiment 1-8, wherein the composition does not comprise an abrasive.

Embodiment 10: An embodiment of any of the embodiments of embodiment 1-9, wherein the composition does not comprise an additional antibacterial agent.

Embodiment 11: An embodiment of any of the embodiments of embodiment 1-10, wherein the composition does not comprise an acid or a base.

Embodiment 12: An embodiment of any of the embodiments of embodiment 1-11, wherein the composition does not comprise a whitening agent.

Embodiment 13: An embodiment of any of the embodiments of embodiment 1-12, wherein the composition does not comprise an additional flavorant.

Embodiment 14: An embodiment of any of the embodiments of embodiment 1-13, wherein the composition does not comprise a colorant.

Embodiment 15: A composition for the removal of dental calculus, the composition consisting of: sorbitol, wherein the concentration of sorbitol in the composition ranges from 50 wt % to 85 wt %; glycerol, wherein the concentration of glycerol in the composition is less than 10 wt %; and water; wherein the composition is a liquid.

Embodiment 16: A method for removing dental calculus from a tooth surface, the method comprising: contacting the tooth surface with the composition of an embodiment of any of the embodiments of embodiment 1-15, and subsequently scaling dental calculus from tooth surface.

Embodiment 17: An embodiment of embodiment 16, wherein the scaling is performed using a hand scaler.

Embodiment 18: An embodiment of embodiment 16, wherein the scaling is performed using an ultrasonic scaler.

Embodiment 19: An embodiment of any of the embodiments of embodiment 16-18, wherein the composition has a temperature from 30° C. to 50° C. during the contacting.

Embodiment 20: An embodiment of embodiment 19, further comprising: heating the composition prior to the contacting.

Embodiment 21: An embodiment of any of the embodiments of embodiment 16-20, wherein the scaling is begun less than 30 minutes after contacting.

### EXAMPLES

The present disclosure will be better understood in view of the following non-limiting examples.

### Example 1. Liquid Formulation of Composition

One exemplary liquid formulation of the provided composition consists of the following components: 70 wt % sorbitol, 5 wt % glycerin, and water.

### Example 2. Gel Formulation of Composition

One exemplary gel formulation of the provided composition consists of the following components: sorbitol at a concentration from 60-80 wt %, glycerin at a concentration less than 10 wt %, xanthan gum at a concentration less than 10 wt %, sodium hydroxide at a concentration less than 1 wt %, benzyl alcohol at a concentration less than 1 wt %, benzoic acid at a concentration less than 1 wt %, sorbic acid at a concentration less than 1 wt %, and water.

While the disclosure has been described in detail, modifications within the spirit and scope of the disclosure will be readily apparent to those of skill in the art in view of the foregoing discussion, relevant knowledge in the art, and references discussed above in connection with the Background and Detailed Description, the disclosures of which are all incorporated herein by reference. In addition, it should be understood that aspects of the disclosure and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure.

What is claimed is:

1. A composition for the removal of dental calculus, the composition consisting essentially of:
sorbitol, wherein the concentration of sorbitol in the composition ranges from 50 wt % to 85 wt %;
glycerol, wherein the concentration of glycerol in the composition is less than 10 wt %; and
water;
wherein the composition is a liquid.

2. The composition of claim 1, wherein the mass ratio of the sorbitol to the glycerol ranges from 5:1 to 50:1.

3. The composition of claim 1, wherein the composition further comprises mannose, and wherein the concentration of mannose in the composition is less than 10 wt %.

4. The composition of claim 1, wherein the composition does not comprise an additional sugar alcohol.

5. The composition of claim 1, wherein the composition does not comprise a metal.

6. The composition of claim 1, wherein the composition does not comprise an additional chelating agent.

7. The composition of claim 1, wherein the composition does not comprise an additional humectant.

8. The composition of claim 1, wherein the composition does not comprise a surfactant.

9. The composition of claim 1, wherein the composition does not comprise an abrasive.

10. The composition of claim 1, wherein the composition does not comprise an additional antibacterial agent.

11. The composition of claim 1, wherein the composition does not comprise an acid or a base.

12. The composition of claim 1, wherein the composition does not comprise a whitening agent.

13. The composition of claim 1, wherein the composition does not comprise an additional flavorant.

14. The composition of claim 1, wherein the composition does not comprise a colorant.

15. A composition for the removal of dental calculus, the composition consisting of:
sorbitol, wherein the concentration of sorbitol in the composition ranges from 50 wt % to 85 wt %;
glycerol, wherein the concentration of glycerol in the composition is less than 10 wt %; and
water;
wherein the composition is a liquid.

16. A method for removing dental calculus from a tooth surface, the method comprising:
contacting the tooth surface with the composition of claim 1, and subsequently scaling dental calculus from tooth surface.

17. The method of claim 16, wherein the scaling is performed using a hand scaler.

18. The method of claim 16, wherein the scaling is performed using an ultrasonic scaler.

19. The method of claim 16, wherein the composition has a temperature from 30° C. to 50° C. during the contacting.

20. The method of claim 19, further comprising:
heating the composition prior to the contacting.
